(19) United States
(12) Patent Application Publication
(10) Pub. No.: US 2025/0009461 A1
(43) Pub. Date: Jan. 9, 2025

(54) SYSTEM FOR DETERMINING IMPLANT-TISSUE CONTACT

(71) Applicant: Spectrumedics Holdings Pte Ltd

(72) Inventors: Jo Ern YEOH; Djunarto NG; Tik Mun Edmund WOO; Xian Wei Jeremy GOH; Hui Qun PHANG

(21) Appl. No.: 18/708,569

(22) PCT Filed: Nov. 9, 2022
(86) PCT No.: PCT/SG2022/050814

§ 371 Date: May 8, 2024

(30) Foreign Application Priority Data
Nov. 10, 2021 (SG) 10202112472S

(51) Int. Cl. A61B 90/00; A61B 5/0538; A61F 2/24

(52) U.S. Cl. CPC A61B 90/06; A61B 5/0538; A61F 2/246; A61B 2090/065; A61F 2220/0016

(57) ABSTRACT

Disclosed herein are implants and systems comprising the implants, where the implants comprise:
a tissue grasping member comprising a switching member, which switching member comprises a tissue contact portion and a first electrically conductive contact portion; and
a connecting member comprising a second electrically conductive contact portion, wherein:
the first electrically conductive contact portion and second electrically conductive contact portion are each configured to be electrically connected to an electrical circuit;
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
   (A) the switch is biased to the closed position and is configured to be opened by contact between the tissue contact portion of the switching member and bodily tissue; or
   (B) the switch is biased to the open position and is configured to be closed by contact between the tissue contact portion of the switching member and bodily tissue.

### FIELD OF THE INVENTION

The present invention relates to a tissue detection system that determines the instance when an implant makes contact with internal body tissue.

### BACKGROUND OF THE INVENTION

Transcatheter valve repair is a procedure where an implant is inserted into the body and placed at the site of a leaky or regurgitating valve. The end result mimics that of standard open heart valve repair, and is considered to be an effective treatment for patients who are precluded from invasive surgery. However, despite satisfactory results, improvements can still be made to the procedure. During positioning of the implant prior to deployment, cardiologists depend primarily upon visual echocardiographic confirmation of its engagement with the valve leaflets before detaching it from the catheter. Slippage and non-engagement despite visual confirmation of engagement have been known to occur, leading to lengthened procedure times and increasing the risk of failure. The need to implant multiple clips is also not uncommon in such procedures due to initial positioning difficulties.

There is therefore a need for a convenient and easy to use tissue detection system that may be integrated into an implant and delivery system. This would complement the primary method of echocardiographic visualization and thus provide a secondary means of ascertaining tissue contact during the deployment procedure.

### BRIEF SUMMARY OF THE INVENTION

The present inventors have surprisingly found that an implant as disclosed herein may overcome some or all of the above problems. The invention therefore provides the following numbered statements.

1. An implant comprising:
a tissue grasping member comprising a switching member, which switching member comprises a tissue contact portion and a first electrically conductive contact portion; and
a connecting member comprising a second electrically conductive contact portion, wherein:
the first electrically conductive contact portion and second electrically conductive contact portion are each configured to be electrically connected to an electrical circuit;
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
   (A) the switch is biased to the closed position and is configured to be opened by contact between the tissue contact portion of the switching member and bodily tissue; or
   (B) the switch is biased to the open position and is configured to be closed by contact between the tissue contact portion of the switching member and bodily tissue.

2. The implant according to statement 1, wherein the tissue grasping member is a leaflet grasping member of a valve repair device, more optionally wherein the valve repair device is suitable for mitral valve edge-to-edge repair and/or tricuspid valve edge-to-edge repair.

3. The implant according to Statement 1 or 2, wherein the switching member is integrally formed with the tissue grasping member.

4. The implant according to any one of the preceding statements, wherein the switching member is a lever.

5. The implant according to Statement 4, wherein the switching member comprises one or more flexible portions, optionally wherein the one or more flexible portions form at least 10% of the length of the switching member, such as at least 20%, at least 30%, at least 40%, at least 50%, at least 60%, at least 70% or at least 80%.

6. The implant according to Statement 5, wherein at least a part of the one or more flexible portions is located at a curved portion of the lever and/or at the first electrically conductive portion.

7. The implant according to Statement 6, wherein the curved portion of the lever comprises a curve of from about 45° to about 135°, such as about 90°.

8. The implant according to any one of Statements 5 to 7, wherein the one or more flexible portions comprise one or more selected from the group consisting of hairpin turns, a zig-zag portion and a laddered portion.

9. The implant according to any one of the preceding statements, wherein the switching member is a lever comprising one or more flexible portions,
wherein:
   the one or more flexible portions comprise one or more selected from the group consisting of hairpin turns, a zig-zag portion and a laddered portion;
   at least a part of the one or more flexible portions is located at a curved portion of the lever; and
   the curved portion of the lever comprises a curve of from about 45° to about 135°.

10. The implant according to Statement 9, or Statement 8 as dependent on Statement 6, wherein said hairpin turns, zig-zag portion and/or laddered portion at a curved portion of the lever are substantially orthogonal to the curve of the curved portion;
more optionally wherein said curved portion comprises a curve of from about 45° to about 135°, such as about 90°.

11. The implant according to any one of the preceding statements, wherein the tissue contact portion of the switching member is integrally formed with the switching member.

12. The implant according to any one of the preceding statements, wherein the connecting member comprises one or more electrically insulating surfaces configured to be in contact with one or more surfaces of the tissue grasping member.

13. The implant according to any one of the preceding statements, wherein the switch is biased to the closed position and is configured to be opened by contact between the tissue contact portion of the switching member and bodily tissue.

14. The implant according to any one of the preceding statements, wherein the tissue contact portion comprises a first part configured to make contact with tissue, and a second part that connects the first portion to the rest of the switching member,
wherein the surface area of the first part configured to make contact with tissue is greater than the cross-sectional area of the second part.

15. A system for implant-tissue contact sensing, comprising:
one or more implant portions each comprising:
   a tissue grasping member comprising a switching member, which switching member comprises a tissue contact portion and a first electrically conductive contact portion; and
   a connecting member comprising a second electrically conductive contact portion, and
an electrical signal source electrically connected to each tissue grasping member and connecting member,
wherein for each of the one or more implant portions:
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
   (A) the switch is biased to the closed position and is configured to be opened by contact between the tissue contact portion of the switching member and bodily tissue; or
   (B) the switch is biased to the open position and is configured to be closed by contact between the tissue contact portion of the switching member and bodily tissue.

16. The system according to Statement 15, wherein for one or more of the one or more implant portions, the tissue grasping member is a leaflet grasping member of a valve repair device, more optionally wherein the valve repair device is suitable for mitral valve edge-to-edge repair.

17. The system according to Statements 15 or 16, wherein for one or more of the one or more implant portions, the switching member is integrally formed with the tissue grasping member.

18. The system according to any one of Statements 16 to 17, wherein for one or more of the one or more implant portions, the switching member is a lever.

19. The system according to any one of Statements 15 to 18, wherein for one or more of the one or more implant portions, the switching member comprises one or more hairpin turns, optionally wherein the switching member comprises hairpin turns along at least 10% of its length, such as at least 20%, at least 30%, at least 40%, at least 50%, at least 60%, at least 70% or at least 80% of its length.

20. The system according to any one of Statements 15 to 19, wherein for one or more of the one or more implant portions, the switching member is a lever comprising one or more hairpin turns,
optionally wherein the lever comprises hairpin turns along at least 10% of its length, such as at least 20%, at least 30%, at least 40%, at least 50%, at least 60%, at least 70% or at least 80% of its length.

21. The system according to Statement 20, wherein one or more of the one or more hairpin turns are located at a curved portion of the lever and/or at the first electrically conductive portion.

22. The system according to Statement 21, wherein said hairpin turns at a curved portion of the lever are substantially orthogonal to the curve of the curved portion;
more optionally wherein said curved portion comprises a curve of from about 45° to about 135°, such as about 90°.

23. The system according to any one of Statements 15 to 22, wherein for one or more of the one or more implant portions, the tissue contact portion of the switching member is integrally formed with the switching member.

24. The system according to any one of Statements 15 to 23, wherein for one or more of the one or more implant portions, the connecting member comprises one or more electrically insulating surfaces configured to be in contact with one or more surfaces of the tissue grasping member.

25. The system according to any one of Statements 15 to 24, comprising one or more interface devices,
wherein each implant portion is electrically connected to an interface device configured to provide an output signal, and where each interface device is configured to provide a change in output signal when a complete electrical circuit is formed between the electrical signal source, interface device and implant portion,
   optionally wherein said output signal comprises one or more selected from the group consisting of a light signal, a sound signal, a haptic signal and a vibrational signal.

26. The system according to any one of Statements 15 to 25, comprising two or more implant portions.

27. The system according to Statement 26, wherein each implant portion is electrically connected to a different interface device.

28. The system according to an one of Statements 15 to 27, wherein the electrical connection between the electrical signal source and tissue grasping member comprises an electrically conductive actuating line for moving the tissue grasping member.

29. The system according to any one of Statements 15 to 28, wherein the switch is biased to the closed position and is configured to be opened by contact between the tissue contact portion of the switching member and bodily tissue.

30. The system according to any one of Statements 15 to 29, wherein the tissue contact portion comprises a first part configured to make contact with tissue, and a second part that connects the first portion to the rest of the switching member,
wherein the surface area of the first part configured to make contact with tissue is greater than the cross-sectional area of the second part.

### BRIEF DESCRIPTION OF THE DRAWINGS

FIG. 1 shows an example of a simple valve repair clip with two articulating leaflet capture members and their corresponding tissue grasping members.

FIG. 2 shows the retention features on a tissue grasping member.

FIG. 3 illustrates the intended use of the tissue grasping member and the outer arm with relation to leaflet tissue.

FIG. 4A shows a detailed view of the components that make up the tissue detection system on a tissue grasping member.

FIG. 4B shows a detailed view of the connecting member.

FIG. 5A shows a schematic depicting the electrical circuit that connects the components of the tissue detection system to one another as well as to the user interface.

FIG. 5B shows a simplified schematic of that shown in FIG. 5A.

FIG. 6 illustrates the operation of the tissue detection system in its resting state.

FIG. 7 illustrates the operation of the tissue detection system in its engaged state, when tissue material has come into contact.

FIG. 8A shows a detailed view of the tissue grasping member as in FIG. 4A, with a variation of the switching member.

FIG. 8B shows a variation of the configuration in FIG. 8A showing a different embodiment of the switching member.

FIG. 8C shows a variation of the configurations in FIG. 8A-C, showing an amalgamation of the shapes of the switching member.

FIG. 9A shows a variation of the configurations in FIG. 8A-D showing a switching member that is non-integrated and separately attached to the inner tissue grasping member.

FIG. shows is an exploded view detailing the connection of the components shown in FIG. 9A.

FIG. 9C shows another method of attaching the separate switching member to the tissue grasping member.

FIG. 10A shows a detailed view of the tissue grasping member in FIG. 4, showing one configuration of the tissue contact portion.

FIG. 10B shows a variation of the configuration in FIG. 10A showing a different embodiment of the tissue contact portion.

FIG. 10C shows a variation of the configurations in FIG. 10A and FIG. 10B showing another different embodiment of the tissue contact portion.

FIG. 11A illustrates how integration of the switching member and tissue contact portion may look.

FIG. 11B shows the assembly in FIG. 11A with the connecting member removed.

FIG. 11C illustrates how the combined switching member and tissue contact portion may look as a separate piece from the tissue grasping member.

FIG. 12A shows a variation of the tissue detection system in which the switch is biased open in its resting state.

FIG. 12B shows a variation of the tissue detection system in which the switch is biased open, when tissue material has come into contact.

FIG. 13 shows a variation of the tissue detection system in which the switching member has a flexible portion.

FIG. 14 shows another variation of the tissue detection system in which the switching member has a flexible portion.

### DETAILED DESCRIPTION

The most prevalent mitral valve repair devices available come in the form of a clip-like structure. The basic principle of mitral valve edge-to-edge repair requires that the trailing edges of the two leaflets of the valve be brought together at the middle and be permanently joined, thus allowing the formation of two adjacent orifices during atrial diastole for anterograde blood flow. The clip structure typically does this by having two outer-actuated arms that capture the ventricular faces of the trailing edges, as well as two inner-actuated grasping members that grip the atrial faces of the trailing edges. Together, the two pairs of grasping members and arms capture each leaflet at their trailing edges and pull them together when the clip structure is closed. Difficulties in this process arise when there is poor visualization through ultrasound, or when leaflet morphology is less than ideal. Physicians have expressed the desire to be able to accurately and without a doubt, determine the instance when the grasping member of a mitral valve repair device has made contact with the trailing edge of a leaflet.

The invention provides an implant comprising:
a tissue grasping member comprising a switching member, which switching member comprises a tissue contact portion and a first electrically conductive contact portion; and
a connecting member comprising a second electrically conductive contact portion, wherein:
the first electrically conductive contact portion and second electrically conductive contact portion are each configured to be electrically connected to an electrical circuit;
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
   (A) the switch is biased to the closed position and is configured to be opened by contact between the tissue contact portion of the switching member and bodily tissue; or
   (B) the switch is biased to the open position and is configured to be closed by contact between the tissue contact portion of the switching member and bodily tissue.

The word “comprising” refers herein may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, reference to “an oxygen carrier” includes mixtures of two or more such oxygen carriers, reference to “the catalyst” includes mixtures of two or more such catalysts, and the like.

The implant of the invention comprises a tissue grasping member. This member serves to grasp tissue, such as a heart valve leaflet. Thus, the tissue grasping member may be a leaflet grasping member of a valve repair device, and may be suitable for mitral valve edge-to-edge repair and/or tricuspid valve edge-to-edge repair.

The tissue grasping member comprises a switching member, which switching member comprises a tissue contact portion and a first electrically conductive contact portion. The implant also comprises a connecting member comprising a second electrically conductive contact portion.

During use, the tissue contact portion of the switching member will make contact with tissue when the implant is placed into the correct position. This contact will cause the switching member to move, and will bring the first electrically conductive contact portion into contact with the second electrically conductive contact portion. Since both the first and second electrically conductive contact portion will, during use, be connected to an electrical circuit, this contact between the first and second electrically conductive contact portion will complete a circuit, and may therefore generate a signal to an operator. This signal allows the operator to know that the implant is in the correct position.

In some embodiments of the invention that may be mentioned herein, the switching member may be integrally formed with the tissue grasping member. This may improve the ease of manufacture, and by reducing the number of connections in the device, the risk of failure may be reduced.

In some embodiments of the invention that may be mentioned herein, the switching member may be a lever.

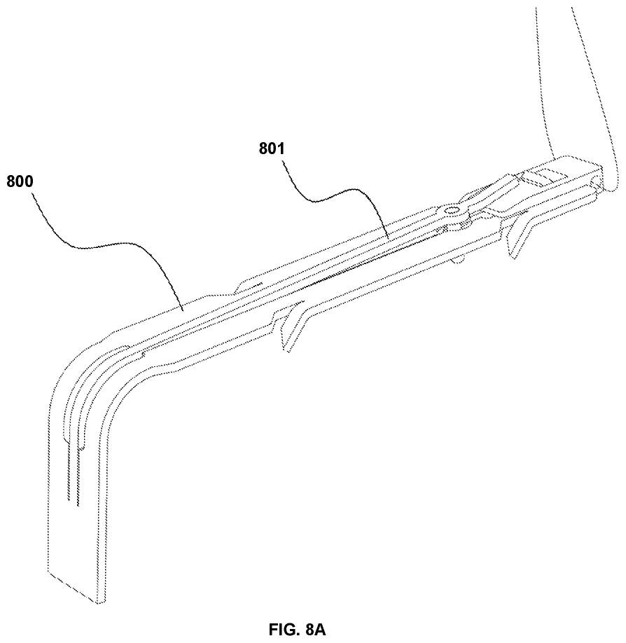

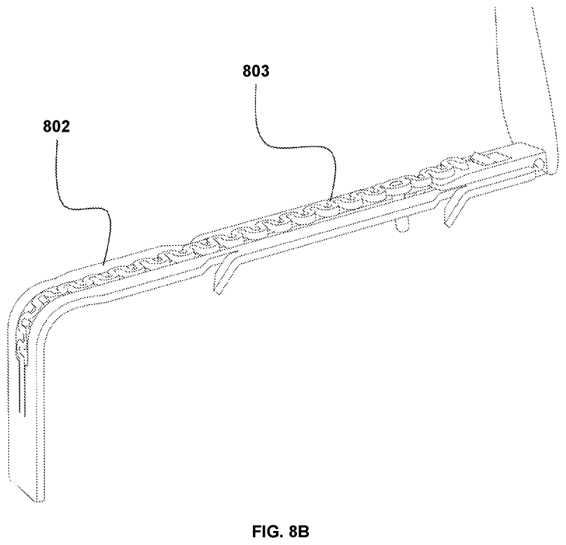

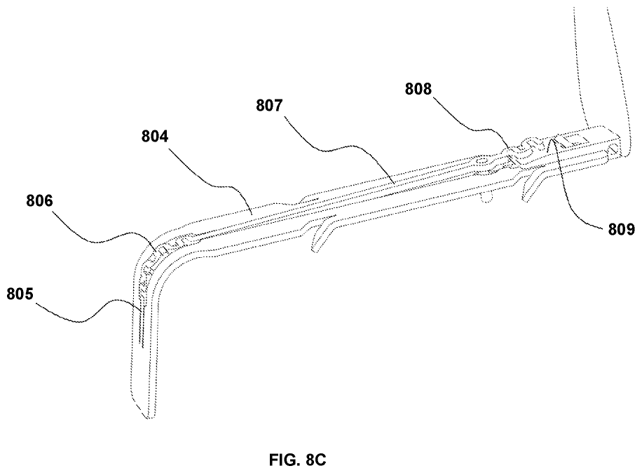

In some embodiments of the invention that may be mentioned herein, the switching member (e.g. lever) may comprise one or more flexible portions. As used in this context, a “flexible portion” is a portion of the switching member that is able to flex or bend more easily (i.e. when subjected to a lower force) than the remainder of the switching member, such that if the switching member flexes or bends, such flexing/bending occurs at the flexible portion. Alternatively, in embodiments in which the “flexible portion” makes up the majority of the switching member (such as in FIG. 8B), the flexible portion may be construed as a portion of the switching member that is able to flex or bend more easily than the remainder of the tissue grasping member. The one or more flexible portions may form at least 10% of the length of the switching member, such as at least 20%, at least 30%, at least 40%, at least 50%, at least 60%, at least 70% or at least 80%.

In some such embodiments of the invention that may be mentioned herein, at least a part of the one or more flexible portions may be located at a curved portion of the lever and/or at the first electrically conductive portion. When the flexible portion is present at a curved portion of the lever, this may improve the flexibility/ease of moving the lever. When the flexible portion is present at the first electrically conductive portion, this may improve the contact between the first and second electrically conductive portions, because the first electrically conductive portion can flex and fully conform to the surface of the second electrically conductive portion.

When the lever comprises a curved portion, the curve may be from about 45° to about 135°, such as about 90°.

Examples of structures/configurations that may be used to form a flexible portion include one or more selected from the group consisting of hairpin turns, a zig-zag portion and a laddered portion, though a skilled person will appreciate that other configurations may be used. When the flexible portion comprises hairpin turns, zig-zag portion and/or laddered portions at a curved portion of the lever, these turns/portions may be substantially orthogonal to the curve of the curved portion. This may serve to improve the flexibility of the switching member/lever.

In some embodiments of the invention that may be mentioned herein, the tissue contact portion of the switching member may be integrally formed with the switching member. This may allow for advantageously straightforward manufacturing.

In some embodiments of the invention that may be mentioned herein, the connecting member may comprise one or more electrically insulating surfaces configured to be in contact with one or more surfaces of the tissue grasping member. This may be advantageous to ensure that an electrical circuit is only formed when the two electrically conductive members are in contact with each other, and that other components making contact does not complete an electrical circuit.

In some embodiments of the invention that may be mentioned herein, the switch formed by the first and second electrically conductive portions may be biased to the closed position and be configured to be opened by contact between the tissue contact portion of the switching member and bodily tissue.

It may be desirable for the tissue contact portion to have a substantial surface area to minimize the pressure imparted onto the tissue (e.g. heart valve). However, it may also be desirable that the switching member is small in size. Therefore, in some embodiments of the invention that may be mentioned herein, the tissue contact portion may comprise a first part configured to make contact with tissue, and a second part that connects the first portion to the rest of the switching member, where the surface area of the first part configured to make contact with tissue is greater than the cross-sectional area of the second part. This advantageously allows for a low pressure to be imported on the tissue, without causing the switching member to be excessively big. Examples of suitable shapes for such embodiments include an L shape and a circular base.

The implant of the invention may be used in a system for implant-tissue contact sensing. Thus, the invention also provides a system for implant-tissue contact sensing, comprising:
one or more implant portions each comprising:
   a tissue grasping member comprising a switching member, which switching member comprises a tissue contact portion and a first electrically conductive contact portion; and
   a connecting member comprising a second electrically conductive contact portion, and
an electrical signal source electrically connected to each tissue grasping member and connecting member,
wherein for each of the one or more implant portions:
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
(A) the switch is biased to the closed position and is configured to be opened by contact between the tissue contact portion of the switching member and bodily tissue; or
(B) the switch is biased to the open position and is configured to be closed by contact between the tissue contact portion of the switching member and bodily tissue.

The features described above in relation to the implant of the invention apply equally to the system of the invention.

The system may be used to provide a signal to an operator during implantation of the implant. For example, the system may comprise one or more interface devices, wherein each implant portion is electrically connected to an interface device configured to provide an output signal, and where each interface device is configured to provide a change in output signal when a complete electrical circuit is formed between the electrical signal source, interface device and implant portion. The output signal may comprise one or more selected from the group consisting of a light signal, a sound signal, a haptic signal and a vibrational signal.

In some embodiments of the invention that may be mentioned herein, the system may comprise two or more implant portions. Each implant portion may be electrically connected to the same, or to a different, interface device. In general, it will be advantageous for each implant portion to be electrically connected to a different interface device to allow an operator to determine which of the implant portions is contacting tissue in the desired manner.

In some embodiments of the invention that may be mentioned herein, the connection between the electrical signal source and tissue grasping member may comprise an electrically conductive actuating line for moving the tissue grasping member.

The invention therefore provides a solution that integrates a mechanically-actuated tissue detection system suitable for the grasping members of mitral valve repair devices, described in more detail below with reference to the drawings.

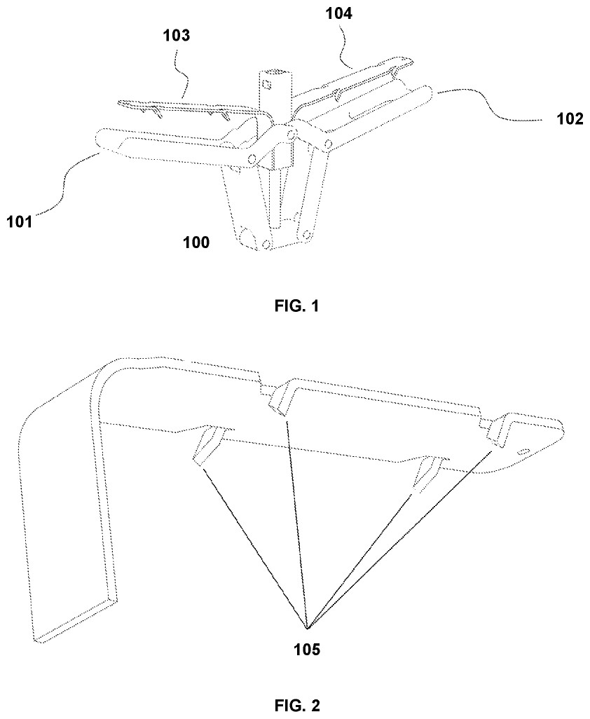

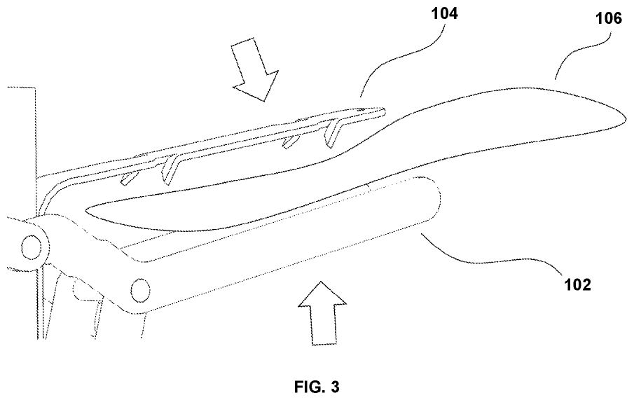

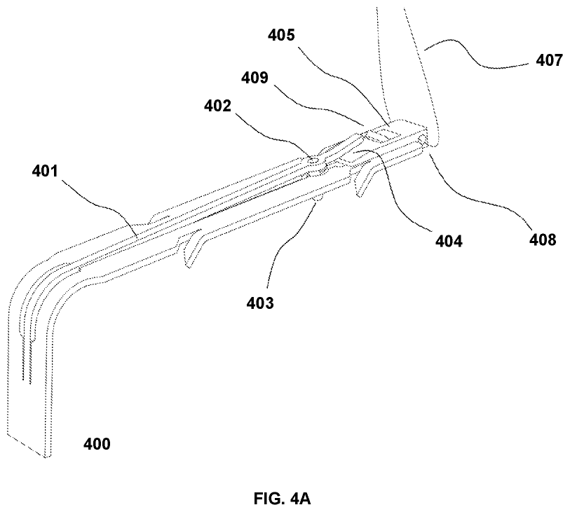

In FIG. 1, a typical design of a mitral valve repair device 100 is shown. 101 and 102 show outer arms while 103 and 104 are inner tissue grasping members. 101, 102, 103 and 104 are typically actuated via control members inside the device that lead to an external handle operated outside of the body during a transcatheter procedure. The pairs 101 and 103, and 102 and 104 each capture the trailing edges of both the anterior and posterior leaflets of the mitral valve. FIG. 2 depicts the grasping member typically having retention features 105 such as hooks or barbs that allow them to easily grip on to the tissue material without slipping away. In some cases, these retention features may instead be found on the arms 101 and 102, or may be found on all four members depending on the design and intended operation of the particular valve repair device. FIG. 3 illustrates how the leaflet tissue 106 of a valve may be intended to be captured by the members 102 and 104.

The concept of the present invention may be implemented with the tissue grasping members of such mitrial valve repair devices. However, one skilled in the art would understand that a similar principle may be applied to the outer arm members, albeit with a slightly different physical implementation.

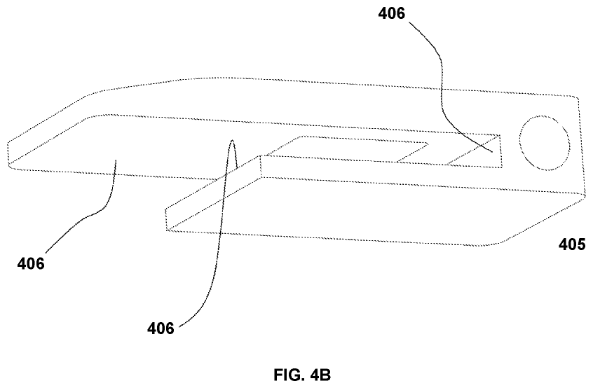

FIG. 4A shows the basic components that make up the tissue grasping member 400. The switching member 401 may be cut out of the material thickness of the tissue grasping member 400 by means of laser-cutting or other suitable means such as drilling or milling. A tissue contact portion 402 is located toward the far end of the switching member 401, and comprises a part 403 configured to make contact with tissue. The far end of the switching member 401 is shown to be resting against a second electrically conductive contact portion 404 of the connecting member 405 sitting at the very tip of the tissue grasping member 400. The collection of surfaces 406 shown in FIG. 4B is electrically-insulated and sits directly in between the connecting member 405 and the electrically-conductive surfaces of the tissue grasping member 400. This collection of surfaces 406 serves to electrically separate the metallic surfaces of the switching member 401 and the metallic surfaces of the connecting member 405. The flow of current thus may only occur when an electrically conductive portion of the switching member 401 is physically touching the electrically conductive portion 404 of the connecting member 405, enabling this pair to function as an electrical switch in a circuit. The electrically-insulated collection of surfaces 406 may be composed of any biocompatible insulating material such as PEEK, parylene, PTFE or silicone. The grasping member actuating line 407 loops through the hole 408 on the connecting member 405. This actuating line 407 is electrically-conductive and is in electrical contact with the connecting member 405. The line 407 may be composed of any biocompatible conductive material such as Nitinol or Stainless Steel. A catch 409 physically holds the connecting member 405 in place on the tissue grasping member 400, and may also be cut and bent out of the material thickness of the tissue grasping member 400.

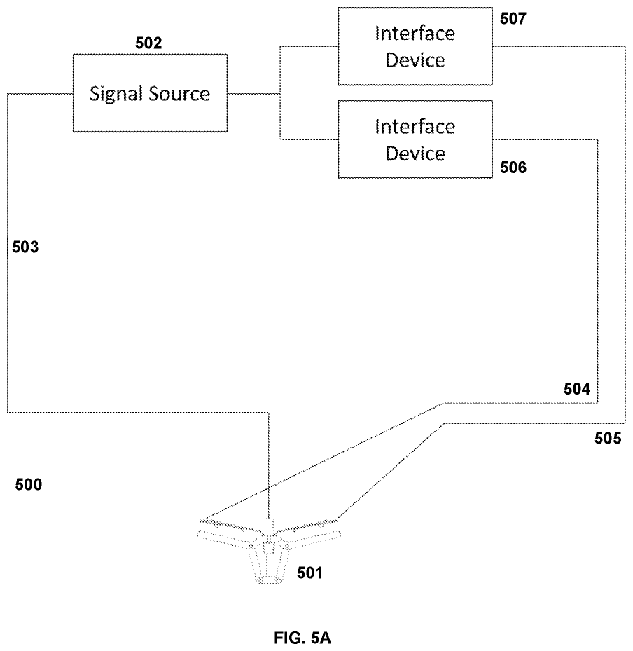

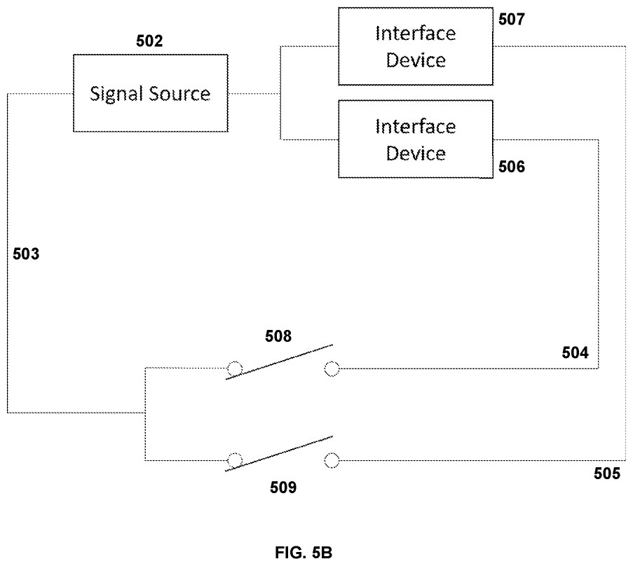

The tissue detection system 500 is shown in its entirety in FIG. 5A, where the implant portion 501 is located within the body. The rest of the system comprises an electrical circuit having a signal source 502, electrically-conductive wires 503, 504 and 505, and interface devices 506 and 507. The signal source 501 may be composed of any suitable device such as a waveform generator or DC power supply. The electrically-conductive wires 503, 504 and 505 may be composed of any suitable biocompatible conductive material such as Nitinol or Stainless Steel. The interface devices 506 and 507 may be any type of device that humans can interact with or receive feedback from, such as a computer, microprocessor, oscilloscope, LCD screen, light-emitting diode, or speaker. The interface devices 506 and 507 are connected in parallel in the circuit, and the tissue grasping members on the mitral valve repair device function as switches which open and close the circuits corresponding to their individual interface devices. A simplified schematic of this is illustrated in FIG. 5B. As the switch 508 is closed, the circuit containing interface device 506 is complete, and the interface device is activated. Similarly, as the switch 509 is closed, the circuit containing interface device 507 is complete, and that interface device is activated. Being in parallel, both may be activated at the same time if both switches are closed at the same time. This allows the user to differentiate between tissue contact on the left and right tissue grasping members individually.

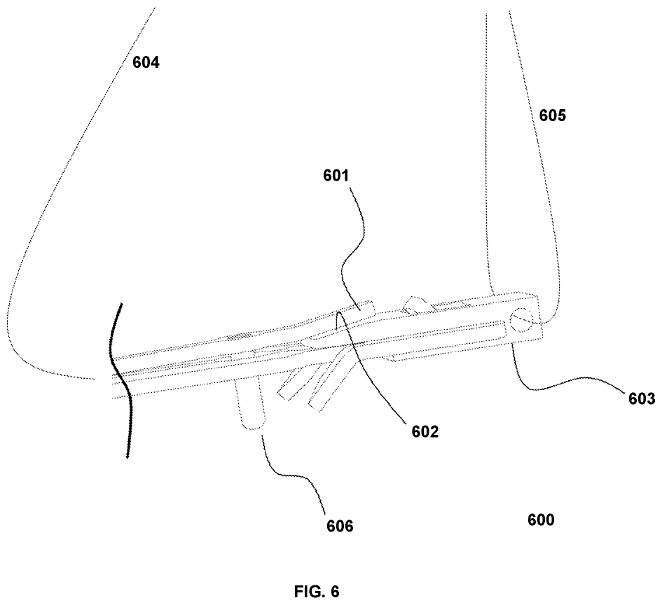

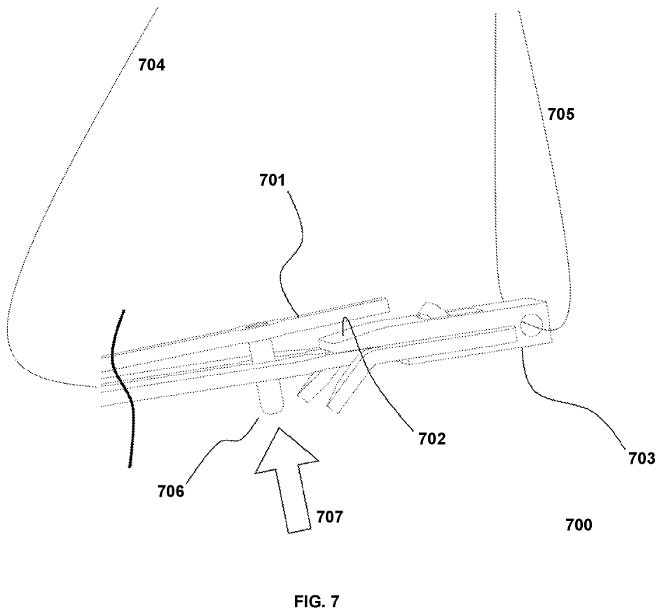

The operation of the tissue detection system is further detailed in FIGS. 6 and 7. In FIG. 6, the implant portion 600 of the tissue detection system is shown to be in its default resting state. The valve repair device is at rest and is not in contact with any tissue. The first electrically conductive portion (of the switching member 601) is resting against the second electrically conductive contact portion 602 of the connecting member 603. The first electrically conductive portion and the second electrically conductive contact portion 602 together form a switch, for example switch 508 as shown in FIG. 5B, in the electrical circuit of the tissue detection system 500. While at rest, the switching member 601 and the second electrically conductive contact portion 602 are touching, and thus complete the electrical circuit. Current is allowed to flow from the signal source through the electrically conductive wires 604, travelling over switching member 601 across the connecting member 603, and eventually returning though 605 to the interface device. Taking for example that the source device may be a simple DC power supply and the interface device connected to this circuit may be a speaker, a complete electrical circuit would cause the speaker to turn on and produce a continuous tone. This indicates that the tissue detection system is not in contact with anything at the area of the tissue contact portion 606.

In FIG. 7, the implant portion 700 of the tissue detection system is shown to be in its engaged state, i.e. there is tissue material that the tissue contact portion 706 has engaged. The downward motion of the tissue grasping member 700 pushes against the tissue material. Consequently, the flexible switching member 701 is opposed by the tissue material with force 707 and flexes upwards, breaking its contact with the second electrically conductive contact portion 702 of the connecting member 703. The electrical circuit is thus broken when the switch is disconnected. Assuming again, a DC power supply for the source device, and a speaker for the interface device, a broken circuit causes the speaker to stop emitting its tone. This indicates to the operator that tissue contact has been made.

Further aspects and embodiments of the invention are described below.

In FIG. 8A a straight beam 801 is cut out of the thickness of the material of the tissue grasping member 800. This is the same configuration as that shown in FIG. 4A. A straight beam is the most basic form of the lever concept, where the length of the beam relative to its thickness allows it to flex to a significant degree out of the plane of the tissue grasping member 800. A variation 803 of the straight beam switching member 801 is shown in FIG. 8B, where instead of going fully straight, the switching member may be cut to different shapes and patterns. In this case, a series of hairpin turns are cut, in order to increase its flexibility relative to the tissue grasping member 802 and enable it to flex with less force applied. FIG. 8C shows a combination of the designs of the straight beam switching member 801 and the hairpin turn switching member 803. By combining both designs, the flexibility of the hybrid switching member 805 can be further tuned and refined to suit the application. For example, the hairpin loop section 806 may be more flexible than the straight beam section 807, which allows the hairpin loop section 806 to flex first, while keeping the straight beam section 807 relatively straight. This may help the switching member 805 keep its conformance to the tissue grasping member 804 as it flexes, and may also help to prevent the switching member 805 from prolapsing to the opposite face of the tissue grasping member. The far-end hairpin loop section 808 may also be more flexible in order for it to flex and rest evenly on the contacting surface 809. This in turn promotes better electrical contact between the two surfaces.

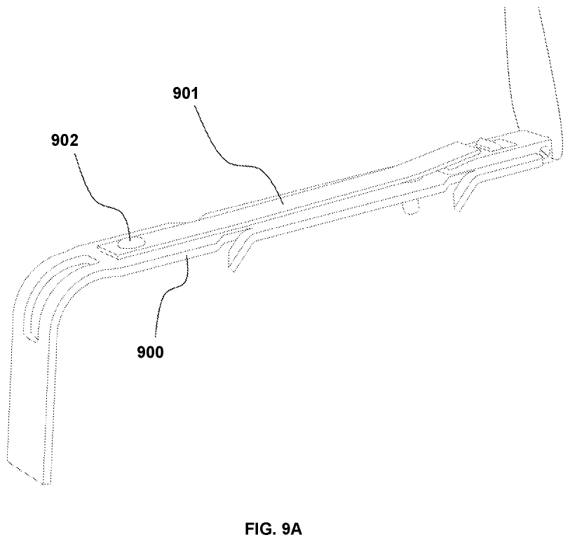

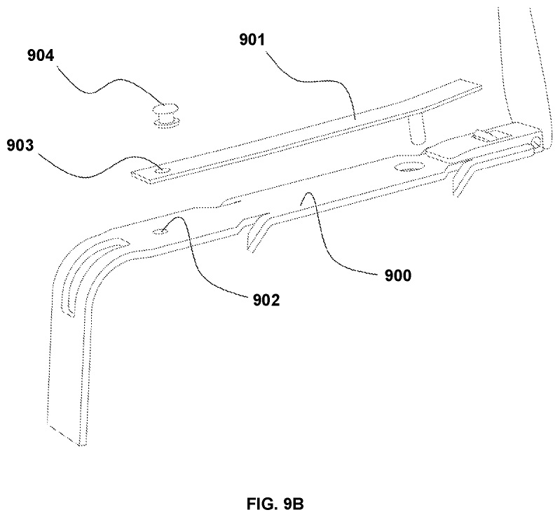

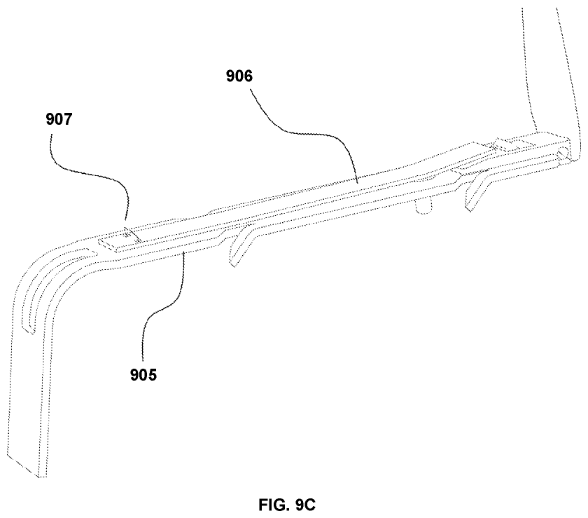

FIG. 9A shows a switching member 901 that is separate from the tissue grasping member 900, and which is attached to the member by mechanical means. By having a separate piece, it becomes possible to better isolate the forces that are acting on each individual component. For example, the flexing of the tissue grasping member 900 may be less likely to affect the flexing of the switching member 901, thus allowing the circuit to remain electrically complete during the initial phase of actuating the tissue grasping members. FIG. 9B illustrates how such a separate switching member 901 may be attached to the tissue grasping member 900. In this instance, both the switching member 901 and the tissue grasping member 900 have mounting holes 902 and 903 respectively, corresponding to each other. The rivet 904 holds the two pieces together securely and allows electrical contact to be established between them. The rivet 904 may or may not be electrically conductive depending on whether or not electrically-insulating surfaces are present either on switching member 901 or tissue grasping member 900. Other methods of attaching the switching member to the tissue grasping member may also be used. Such an example is shown in FIG. 9C, where the switching member 906 and tissue grasping member 905 have another set of matching mounting holes. This allows the suture wire 907 to be looped through the sets of holes in order to securely fasten the two pieces together. Besides suture wire, wires or lines of other materials may also be used, including but not limited to, polyesters, PEEK, Nitinol or Stainless Steel.

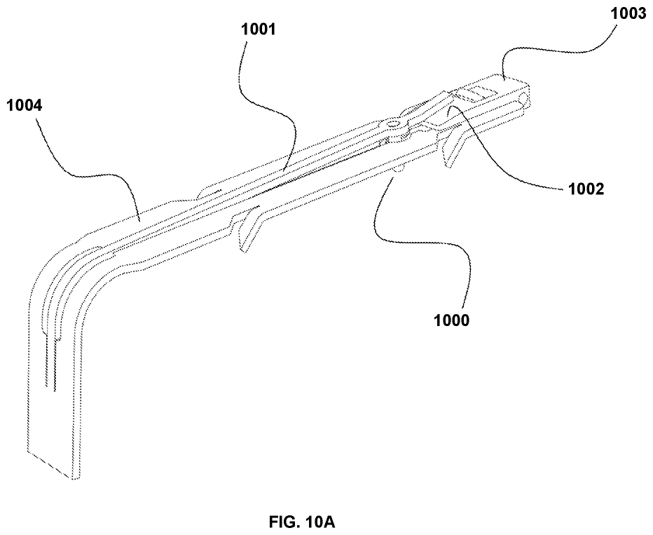

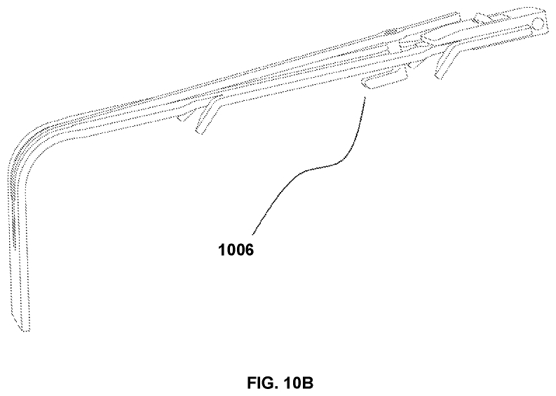

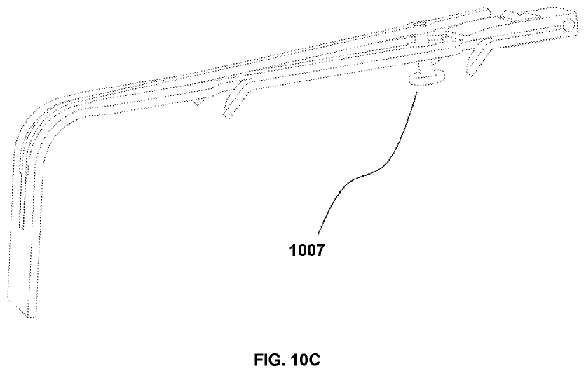

In FIG. 10A, the tissue contact portion 1000 takes the form of a straight cylindrical pin, much like that described in FIG. 4A. This tissue contact portion is responsible for transferring the force of contact from the tissue material to the switching member 1001, enabling the switching member 1001 to break physical and electrical contact from the second electrically conductive contact portion 1002 of the connecting member 1003. The height of the tissue contact portion 1000 may also be varied so as to partly tune the sensitivity of the tissue detection system to tissue materials in the body. For example, a longer pin may allow the tissue detection system to more quickly determine if tissue contact has been made, but at the same time, could also lead to false positive readings if the switching member 1001 has already lifted off from the second electrically conductive contact portion 1002 of the connecting member 1003 before the retention features of the tissue grasping member 1004 have managed to securely grip the tissue material. Conversely, too short a pin could lead to false negatives. Aside from varying the height of the tissue contact portion, the shape may also be varied in order to better tune the sensitivity of the tissue detection system. FIG. 10B shows another configuration of the tissue contact portion 1006 where instead of a straight cylindrical pin, it may take the form of an L-shaped post. The longer base of the L-shaped tissue contact portion 1006 allows the pressure from the tissue material to be distributed over a larger area, thus lowering the chance that a singular point pin pierces the tissue material. This configuration may also be advantageous in cases where space is limited on the tissue grasping member. FIG. 10C shows yet another configuration of the tissue contact portion 1007, where a circular base is used. This may allow for an even greater distribution of pressure across the tissue material, given that enough space is available on the tissue grasping member. These variations demonstrate that any shape of tissue contact portion may be considered in order to tune the pressure exerted upon the tissue, and those shown above are not exhaustive.

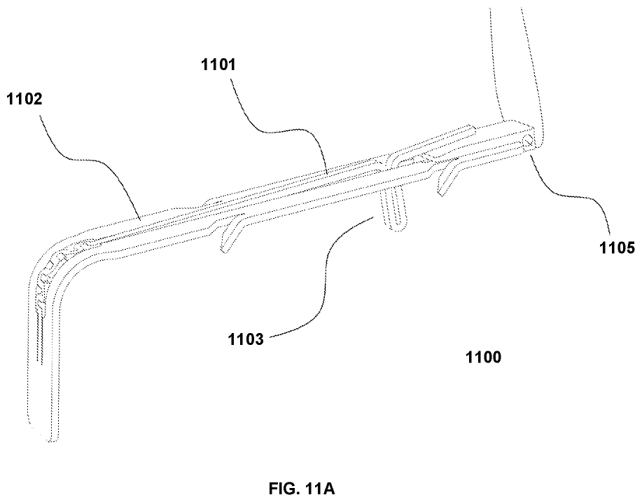

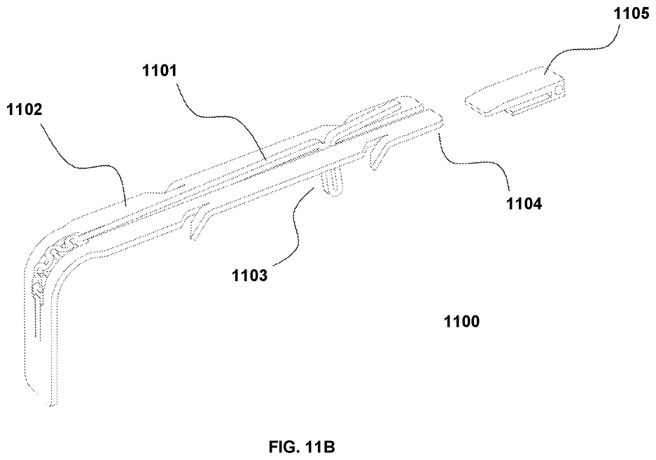

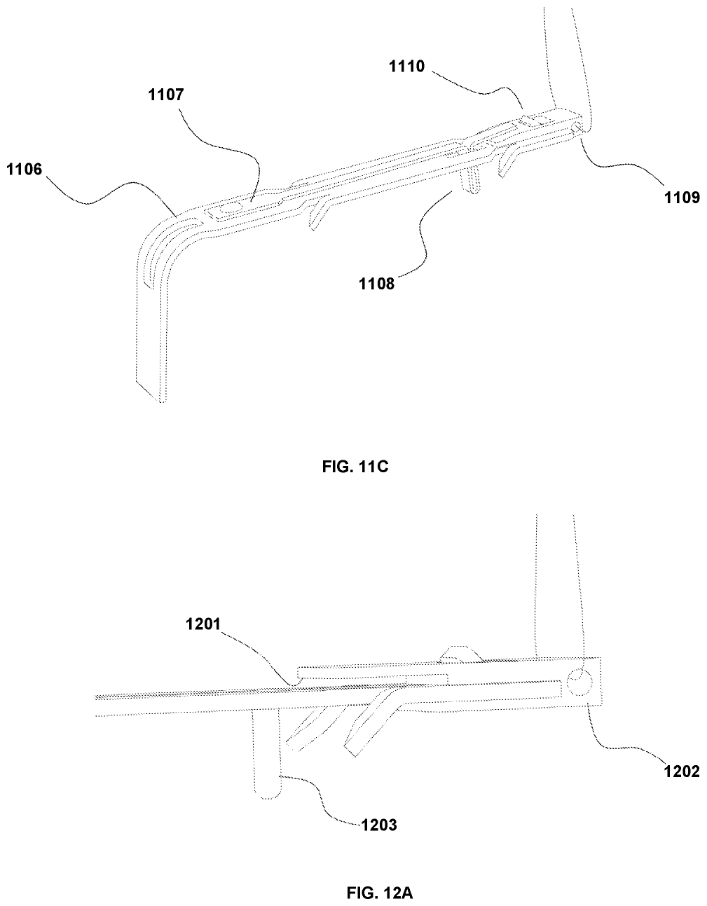

In some cases, it may also be desirable to integrate the switching member and tissue contact portion into one contiguous structure, so as to reduce the complexity and number of separate components to the tissue detection system. FIG. 11A and FIG. 11B show one such example of a system 1100. The entire structure may be cut from a single sheet of leaflet grasper member material, and bent to form the final switching member 1101. The pinched section 1103 may now serve a function as the tissue contact portion, thus eliminating the need for the assembly of two separate components. This however, necessitates that the far end 1104 of the tissue grasping member 1102 now be split down the middle, in order to achieve the length required to produce the pinched section 1103. As such, the connecting member 1105 may now serve the function of linking the two separated pieces of the tissue grasping member 1102 when it is attached. FIG. 11C shows the same concept applied to a separate switching member 1107 and tissue grasping member 1106. The switching member 1107 may again be cut and shaped from a single sheet of material, where the pinched section 1108 serves a function as the tissue contact portion. The tissue grasping member 1106 may then have mounting holes for attaching the switching member 1107, as well as a hole for allowing the pinched section 1108 to pass through to make contact with tissue material. In this case, there is no need for the tissue grasping member 1106 to be split down the middle as the switching member may be made from a separate piece of material. As such, the same method of attaching the connecting member 1109 using the catch 1110 may be used.

FIG. 12A shows a variation of the tissue detection system in which the switch is biased open in its resting state. The connecting member 1202 has a gap between itself at the second electrically conductive contact portion 1201 and the straight beam switching member. The gap ensures that the electrical circuit remains open at rest while the tissue contact portion 1203 is not in contact with any tissue. Upon contact with tissue (FIG. 12B), the tissue contact portion 1203 would be pushed upon by the tissue, and the straight beam switching member would be displaced upwards. The distal end of the straight beam switching member would then come into contact with the contacting surface 1201, thus establishing physical and electrical continuity. The electrical circuit is closed and current is allowed to flow. This would then be indicated on the interface devices along the circuit that tissue contact has occurred.

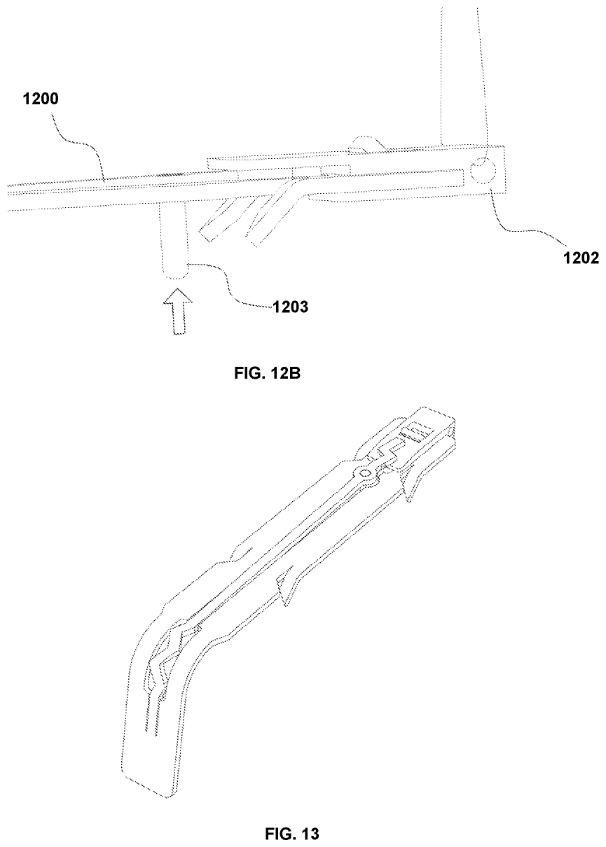

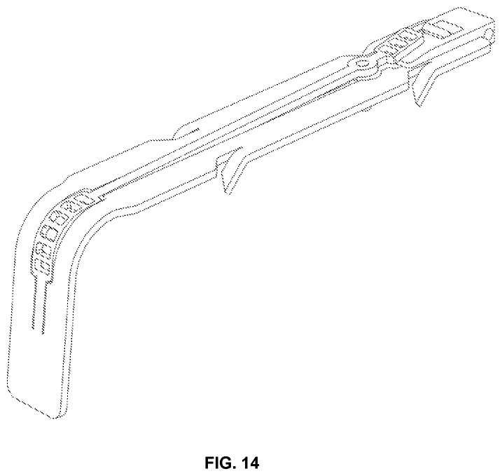

FIGS. 13 and 14 show variations of the embodiments of the invention that are shown in FIGS. 8B and 8C, except the hairpin turns at the curved portion of the switching member are replaced by a zig-zag portion (FIG. 13) or a laddered portion (FIG. 14).

1. An implant comprising:
a tissue grasping member comprising a switching member, which switching member comprises a tissue contact portion and a first electrically conductive contact portion; and
a connecting member comprising a second electrically conductive contact portion, wherein:
the first electrically conductive contact portion and second electrically conductive contact portion are each configured to be electrically connected to an electrical circuit;
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
(A) the switch is biased to the closed position and is configured to be opened by contact between the tissue contact portion of the switching member and bodily tissue; or
(B) the switch is biased to the open position and is configured to be closed by contact between the tissue contact portion of the switching member and bodily tissue.

2. The implant according to claim 1, wherein the tissue grasping member is a leaflet grasping member of a valve repair device.

3. The implant according to claim 1, wherein the switching member is integrally formed with the tissue grasping member.

4. The implant according to claim 1, wherein the switching member is a lever.

5. The implant according to claim 4, wherein the lever comprises one or more flexible portions.

6. The implant according to claim 5, wherein at least a part of the one or more flexible portions is located at a curved portion of the switching member and/or at the first electrically conductive portion.

7. The implant according to claim 6, wherein the curved portion of the lever comprises a curve of from about 45° to about 135°.

8. The implant according to claim 5, wherein the one or more flexible portions comprise one or more selected from the group consisting of hairpin turns, a zig-zag portion and a laddered portion.

9. The implant according to claim 1, wherein the switching member is a lever comprising one or more flexible portions, wherein:
the one or more flexible portions comprise one or more selected from the group consisting of hairpin turns, a zig-zag portion and a laddered portion;
at least a part of the one or more flexible portions is located at a curved portion of the lever; and
the curved portion of the lever comprises a curve of from about 45° to about 135°.

10. The implant according to claim 9, wherein said hairpin turns, zig-zag portion and/or laddered portion at a curved portion of the lever are substantially orthogonal to the curve of the curved portion.

11. The implant according to claim 1, wherein the tissue contact portion of the switching member is integrally formed with the switching member.

12. The implant according to claim 1, wherein the connecting member comprises one or more electrically insulating surfaces configured to be in contact with one or more surfaces of the tissue grasping member.

13. The implant according to claim 1, wherein the switch is biased to the closed position and is configured to be opened by contact between the tissue contact portion of the switching member and bodily tissue.

14. The implant according to claim 1, wherein the tissue contact portion comprises a first part configured to make contact with tissue, and a second part that connects the first portion to the rest of the switching member,
wherein the surface area of the first part configured to make contact with tissue is greater than the cross-sectional area of the second part.

15. A system for implant-tissue contact sensing, comprising:
one or more implant portions each comprising:
a tissue grasping member comprising a switching member, which switching member comprises a tissue contact portion and a first electrically conductive contact portion; and
a connecting member comprising a second electrically conductive contact portion, and
an electrical signal source electrically connected to each tissue grasping member and connecting member,
wherein for each of the one or more implant portions:
the first electrically conductive contact portion of the switching member and the second electrically conductive contact portion of the connecting member together form a switch having a closed position when the first electrically conductive contact portion of the switching member is in contact with the second electrically conductive contact portion of the connecting member, and an open position when the first electrically conductive contact portion of the switching member is not in contact with the second electrically conductive contact portion of the connecting member, and either:
(A) the switch is biased to the closed position and is configured to be opened by contact between the tissue contact portion of the switching member and bodily tissue; or
(B) the switch is biased to the open position and is configured to be closed by contact between the tissue contact portion of the switching member and bodily tissue.

16. The system according to claim 15, wherein for one or more of the one or more implant portions, the tissue grasping member is a leaflet grasping member of a valve repair device.

17. The system according to claim 15, wherein for one or more of the one or more implant portions, the switching member is integrally formed with the tissue grasping member.

18. The system according to claim 16, wherein for one or more of the one or more implant portions, the switching member is a lever.

19. The system according to claim 15, wherein for one or more of the one or more implant portions, the switching member comprises one or more hairpin turns.

20. The system according to claim 15, wherein for one or more of the one or more implant portions, the switching member is a lever comprising one or more hairpin turns.

21. The system according to claim 20, wherein one or more of the one or more hairpin turns are located at a curved portion of the lever and/or at the first electrically conductive portion.

22. The system according to claim 21, wherein said hairpin turns at a curved portion of the lever are substantially orthogonal to the curve of the curved portion.

23. The system according to claim 15, wherein for one or more of the one or more implant portions, the tissue contact portion of the switching member is integrally formed with the switching member.

24. The system according to claim 15, wherein for one or more of the one or more implant portions, the connecting member comprises one or more electrically insulating surfaces configured to be in contact with one or more surfaces of the tissue grasping member.

25. The system according to claim 15, comprising one or more interface devices,
wherein each implant portion is electrically connected to an interface device configured to provide an output signal, and where each interface device is configured to provide a change in output signal when a complete electrical circuit is formed between the electrical signal source, interface device and implant portion.

26. The system according to claim 15, comprising two or more implant portions.

27. The system according to claim 26, wherein each implant portion is electrically connected to a different interface device.

28. The system according to claim 15, wherein the electrical connection between the electrical signal source and tissue grasping member comprises an electrically conductive actuating line for moving the tissue grasping member.

29. The system according to claim 15, wherein the switch is biased to the closed position and is configured to be opened by contact between the tissue contact portion of the switching member and bodily tissue.

30. The system according to claim 15, wherein the tissue contact portion comprises a first part configured to make contact with tissue, and a second part that connects the first portion to the rest of the switching member,
wherein the surface area of the first part configured to make contact with tissue is greater than the cross-sectional area of the second part.
